(19) United States
(12) Patent Application Publication
(10) Pub. No.: US 2023/0218973 A1
(43) Pub. Date: Jul. 13, 2023

(54) SYSTEMS AND METHODS FOR ASSISTING PHYSICAL EXERCISES

(71) Applicant: WISTRON CORPORATION, New Taipei City (TW)

(72) Inventors: Yi Hung TUNG, New Taipei City (TW); Ping Hsin LIU, New Taipei City (TW); Ming Chih YEN, New Taipei City (TW); Tai Hsuan CHIU, New Taipei City (TW); Siao Yun YANG, New Taipei City (TW)

(73) Assignee: WISTRON CORPORATION, New Taipei City (TW)

(21) Appl. No.: 17/573,003

(22) Filed: Jan. 11, 2022

(51) Int. Cl. A63B 71/06; G06F 3/04847; G06F 3/0488

(52) U.S. Cl. CPC A63B 71/0622; G06F 3/04847; G06F 3/0488; A63B 2071/0694; A63B 2214/00; A63B 2225/74

(57) ABSTRACT

A physical exercise assistant system and method are disclosed. The physical exercise assistant system includes a pattern generator, a pattern marker, and a user interface. The pattern generator is configured to generate a plurality of geometric patterns. The pattern marker is coupled to the pattern generator and configured to produce a first one of the plurality of geometric patterns on a ground. The user interface is configured to display the plurality of geometric patterns and receive an input for selecting the first one of the plurality of geometric patterns.

### BACKGROUND

The present disclosure relates generally to physical exercise equipment, and, more particularly, to systems and methods for assisting physical exercises.

For people facing mobility challenges which can be anything from knee or hip osteoarthritis, to multiple sclerosis and stroke, an exoskeleton robotic device is often used to help the patient stand and walk during rehabilitation.

With the assistant devices, a physical therapist can direct a patient to perform various exercises for rehabilitation. One of such exercises is to walk along a patterned path with many turns. Conventionally, the patterned paths are either permanently painted on a ground like a playground hopscotch or marked out on a ground using painter's tapes. Such conventional marking methods, however, are less flexible and cannot response to users' movements.

As such, dynamically adjustable systems and methods for assisting physical exercises by those physically impaired people are desired.

### SUMMARY

Accordingly, the embodiments of the disclosure provide a physical exercise assistant system and a physical exercise assistant method.

In an embodiment of the disclosure, the physical exercise assistant system includes a pattern generator, a pattern marker, and a user interface. The pattern generator is configured to generate a plurality of geometric patterns. The pattern marker such as a video projector is coupled to the pattern generator and configured to produce a first one of the plurality of geometric patterns on a ground. The user interface is coupled to the pattern generator and configured to display the plurality of geometric patterns and receive an input for selecting the first one of the plurality of geometric patterns.

In embodiments, the plurality of geometric patterns is stored in a storage unit. Each one of the plurality of geometric patterns has a set of parameters that can be changed through the user interface. The set of predetermined parameters includes at least one of pattern category, pattern size, pattern position, pattern number and pattern color. Any change in the parameters can instantly invoke changes in the produced geometric pattern on the ground so that a user can dynamically adjusting the produced geometric pattern for different exercises.

In embodiments, the user interface is a mobile device with a touch panel display on which a user can create new geometric patterns for the pattern generator. A database may be employed to store an identification code and parameters associated with each of the plurality of geometric patterns, wherein an individual geometric pattern and its associated parameters can be selected by selecting the identification code.

In embodiments, a motion sensor may be employed for detecting a user's location and movement within a produced geometric pattern on the ground and providing the user's location and movement data to the pattern generator and the user interface. In turn, the user's location is marked in the produced geometric pattern.

In embodiments, the user's movement data may be used to modify the parameters associated with the produced geometric pattern or select a different geometric pattern to produce.

In an embodiment of the disclosure, the physical exercise assistant method includes providing a user interface to display a plurality of geometric patterns, selecting a first one of the plurality of geometric patterns on the user interface, and producing the first one of the plurality of geometric patterns on a ground.

In an embodiment of the disclosure, the physical exercise assistant system includes a central controller, a pattern marker, a user interface, and a motion sensor. The central controller is configured to generate a plurality of geometric patterns. The pattern marker is coupled to the central controller and configured to produce a first one of the plurality of geometric patterns on a ground. The mobile controller is configured to display the plurality of geometric patterns and receive an input for selecting the first one of the plurality of geometric patterns. The motion sensor is coupled to the central controller and configured to detect an object location and movement within the produced one of the plurality of geometric patterns and provide the object location and movement data to the central controller and the mobile controller.

### BRIEF DESCRIPTION OF THE DRAWING

FIG. 1 illustrates an assisted exercise system according to embodiments of the present disclosure.

FIG. 2 illustrates exemplary functional modules in the central controller shown in FIG. 1.

FIG. 3 illustrates an components of interface module of the mobile controller shown in FIG. 1.

FIG. 4 illustrates various function components of the pattern generator shown in FIG. 2.

FIG. 5 illustrates an interface display of the mobile controller shown in FIG. 1.

FIG. 6 is a flow-chart illustrating an operation of the assisted exercise system according to embodiments of the present disclosure.

FIG. 7 is a block diagram illustrating a first exemplary operation of the assisted exercise system shown in FIG. 1.

FIG. 8 is a block diagram illustrating a second exemplary operation of the assisted exercise system shown in FIG. 1.

FIG. 9 is a flowchart illustrating a process of using the assisted exercise system shown in FIG. 1.

The drawings accompanying and forming part of this specification are included to depict certain aspects of the disclosure. A clearer conception of the disclosure, and of the components and operation of systems provided with the disclosure, will become more readily apparent by referring to the exemplary, and therefore non-limiting, embodiments illustrated in the drawings, wherein like reference numbers (if they occur in more than one view) designate the same elements. The disclosure may be better understood by reference to one or more of these drawings in combination with the description presented herein.

### DESCRIPTION

The present disclosure relates to systems and methods for assisting physical exercises. Preferred embodiments of the present disclosure will be described hereinafter with reference to the attached drawings.

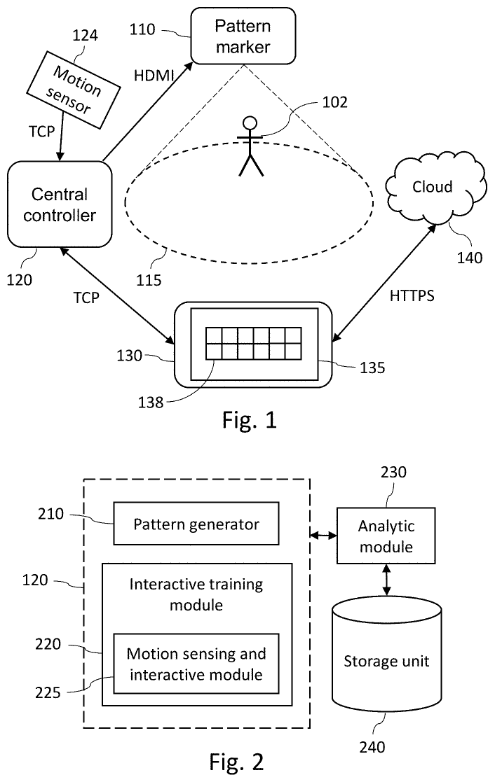

FIG. 1 illustrates an assisted exercise system according to embodiments of the present disclosure. The assisted exercise system includes a pattern marker 110 and a central controller 120, and the pattern marker 110 is coupled to the central controller 120 and configured to produce a plurality of geometric patterns on a ground. In an embodiment, the pattern marker 110 is configured to dynamically generate a pattern 115 on a ground for a patient 102 to exercise upon. As an example, the pattern marker 110 is implemented with a video projector. As another example, the pattern marker 110 is implemented with a LED-embedded carpet. The LEDs are placed in a grid. By selectively turning on the LEDs, different patterns can be generated. The pattern marker 110 receives image data from a central controller 120 through an exemplary HDMI cable. Alternatively, the image data can also be transmitted by a Wi-Fi transmitter and receiver.

Referring again to FIG. 1, the central controller 120 dynamically generates data of the pattern 115 and receives movement data of the patient 102 through a motion sensor 124. In an example, the motion sensor 124 is implemented with a 3-D laser scanning device, radar, or Lidar. The motion sensor 124 is configured to detect the location and movement of the patient 120 within a produced geometric pattern on the ground. In another example, the motion sensor 124 is implemented with a matrix of contact sensor placed on the ground. As shown in FIG. 1, the motion sensor 124 is connected to the central controller 120 by a TCP/IP socket connection. The central controller 120 is coupled to a user interface 138 accessible on, for example, a mobile controller 130, through an exemplary TCP/IP socket connection. The mobile controller 130 is used by an operator to perform various training functions described hereinbelow. The operator can be a physical therapist (trainer) or the patient 102 himself or herself or other people. In an embodiment, the user interface 138 may be on the mobile controller 130 with a touch panel display 135 for interfacing with the operator. The user interface 138 can be used to enter training plans or to provide training instructions. For example, an operator can enter exercise patterns through the user interface 138, and once an exercise session has started, the user interface 138 can display a current location and an intended next location to step into. The user interface 138 can also generate audio instructions based on the current location and a stored exercise program. The mobile controller 130 may be exemplarily coupled to the edge computing system or cloud computing system through the HTTPS protocol for data storage and other computational needs such as exercise routing optimization.

The assisted exercise system shown in FIG. 1 employs interactive and editable graphics projection. Physical therapists use the system to generate graphics and project them to the ground. The graphics are mainly for patients to perform walking or jumping rehabilitation. The graphics can be customized according to the patient's condition. After setting the graphics, the user can interact with the graphics. The physical therapist can use the mobile controller 130 to click on a representative display of the graphics to guide the patient's next actions. After stepping on the projected graphics on the ground, the system will detect and record the patient's location and movement, for instance the knee height, the hips angle, the center of gravity, and the moving speed, through the motion sensor 124. The system also analyzes patients' records and recommend more customized graphics in the future. For instance, when a patient makes progress after a few exercise sessions, the assisted exercise system will recommend more challenging graphics for the patient.

FIG. 2 illustrates exemplary functional modules in the central controller 120 shown in FIG. 1. The central controller 120 includes a pattern generator 210 and an interactive training module 220. The pattern marker 110 is coupled to the pattern generator 210. The pattern generator 210 provides graphic editing function to generate a plurality of various geometric patterns 115 for directing a patient to walk in a certain route in an exercise session. In an embodiment, the pattern generator 210 is configured to alter a predetermined parameter of geometric patterns. As different patients have different physical conditions, the pattern generator 210 allows the predetermined parameters, such as the size, position, repetition, and color, of the generated geometric patterns 115 to be editable in response to different patients' need. In an example, the predetermined parameter is selected from a set consisting of at least one of pattern category, pattern size, pattern position, pattern number and pattern color. In an embodiment, such selection is made through the user interface 138. For example, a user input such as a tap on the touch sensing display 135 can invoke an instant change in the predetermined parameter, i.e., changing from parameter to another of the plurality of geometric patterns. Such parameters belonging to each patient are stored in the central controller 120 and may be transferred to the mobile controller 130 through TCP/IP socket connection. In an embodiment, such parameters are also stored in the cloud 140 by the individual patient's Identification codes. In an embodiment, the analytic module 230 is configured to switch the identification code to be associated with the geometric patterns at least partially based on the patient's movement data.

Referring again to FIG. 2, the interactive training module 220 includes a motion sensing and interactive module 225 which receives patient's location and motion data from the motion sensor 124. The motion sensor 124 may provide the patient's location and movement data to the pattern generator 210 and the user interface 138. The interactive training module 220 compares the generated geometric pattern with the patient's location and motion data to generate positive feedback to the patient and alter the geometric pattern based on predetermined rules stored in the central controller 120. As an example, the feedback may include an audio encouraging prompt such as “right-foot steps forward” or “good jobs”. In response to the motion data, the geometric pattern may be altered to generate a visual prompt for the patient.

Referring again to FIG. 2, the central controller 120 is coupled to analytic module 230 which is in turn coupled to a storage unit 240 for storing a database that contains pattern and training records. In other words, the geometric patterns are stored in the storage unit 240. In an embodiment, the analytic module 230 is configured to modify the parameters associated with geometric patterns at least partially based on the patient's movement data. The analytic module 230 analyses an individual patient's exercise history and generate a recommendation for effective lesson plan through AI technology based thereupon. The analytic module 230 is configured to analyze the individual data, which may include location data and/or movement data, to provide the recommendation. The analytic module 230 also take in an operator's inputs as training settings. Each individual patient has an entry in the database stored in the storage unit 240 to store his or her training history and settings. In this way, the AI analytic module 230 can provide training strategy individually. The analytic module 230 and the storage unit 240 may reside in the central controller 120 or the cloud 140.

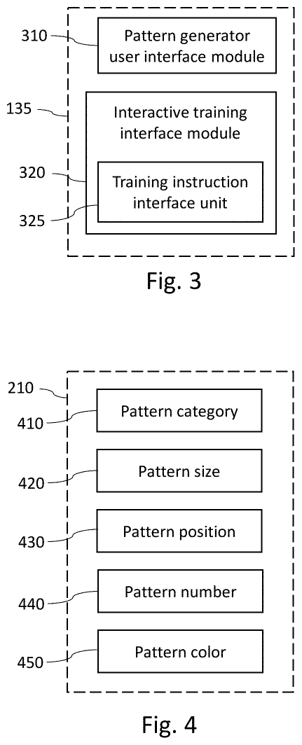

FIG. 3 illustrates an components of interface module of the mobile controller 130 shown in FIG. 1. The mobile controller 130 has a pattern generator user interface module 310 and an interactive training interface module 320 together control and receive operator input from the touch sensing display 135 of the mobile controller 130. The pattern generator user interface module 310 allows an operator to select pre-stored patterns or create new ones. In an example, the user interface is configured to create new geometric patterns for the pattern generator 210 and indicated the training instruction marked in the produced geometric pattern. The interactive training interface module 320 combines a display pattern with a patient's current location and motion data and allows the operator to dynamically alter the pattern and/or generate training instructions. The training instructions are generated through a training instruction interface unit 325 within the interactive training interface module 320. The training instruction interface unit 325 may present a list of prestored instructions or allow the operator to enter new ones in either audio or video form.

FIG. 4 illustrates various functional components of the pattern generator 210 shown in FIG. 2. The functional components include at least one of pattern category 410, pattern size 420, pattern position 430, pattern number 440 and pattern color 450. The pattern category 410 exemplarily includes square trail, hopscotch, nine square grid, wide-and-narrow path, turning path, directional plate, river stones, dance machine or any other geometric graphics, and is selectable. The pattern size 420 refers to unit size of each of the geometric graphics. Different patients may have different stride length thus the pattern size 420 should be different and customizable. The pattern position 430 refers to locations of each of the geometric graphics. It is also customizable to cater to different patients in different poor control. Such as some patients walk straight lines, so that the pattern position 430 is designed in a linear fashion; and other patients need to walk with many turns, so that the pattern position 430 is designed in a complicated 2-dimensional fashion. The pattern number 440 refers to a number of the geometric graphics, which is also customizable based on a patient's physical condition. A stronger patient may be given a higher pattern 440 and vice versa. The pattern color 450 refers to colors of each of the geometric graphics. It is based on the environment, pattern maker specification, or personal preferences. In an embodiment, geometric graphics corresponding to past steps may be given a different color from the geometric graphics corresponding to next steps.

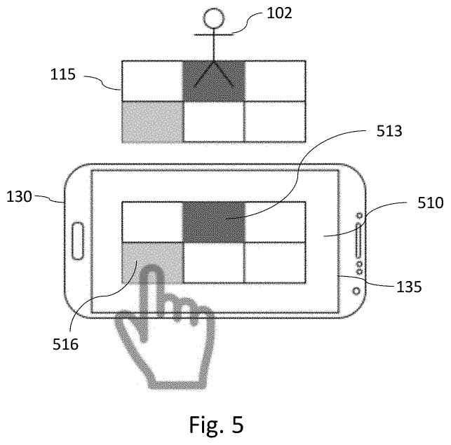

FIG. 5 illustrates an interface display 135 of the mobile controller 130 shown in FIG. 1. The interface display 135 exemplarily displays a rectangular pattern 510 having six rectangular boxes. The same pattern 510 is generated by the pattern marker 110 on the ground as the pattern 115. The patient 120's location is marked in the geometric patterns. A darker shaded box 513 represents the patient 102's current location and a lighter shaded box 516 represents a next location the patient 102 is supposed to step to. An operator such as a physical therapist can click on the interface display 135 and generate a guidance prompt such as turning a particular box into a lighter shaded box, which can guide the patient's next step position.

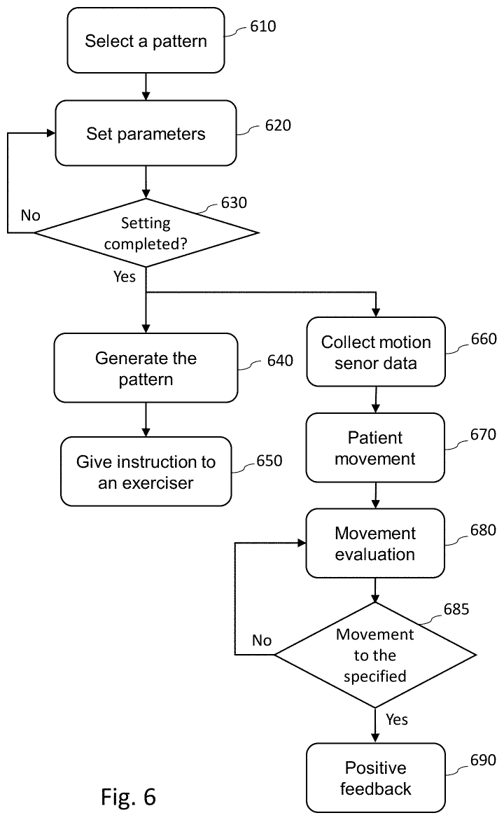

FIG. 6 is a flow-chart illustrating an operation of the assisted exercise system according to embodiments of the present disclosure. The operation begins with block 610 where a pattern is selected on the mobile controller 130. In block 620, the operator sets parameters associated with the pattern. In block 630, the mobile controller 130 waits for the setting to be completed. Once the operator signals the completion of the parameter setting, the process enters block 640 where a pattern is generated on the ground by central control 120. In block 650, the operator gives instruction to the patient either verbally or through prompts on the pattern. In an embodiment, the patient may operate the mobile controller 130 to direct his or her own exercise. However, the patterns and associated instructions may be pre-entered by a physical therapist.

Referring again to FIG. 6, once the parameters are all set in block 630, the motion sensor 124 becomes to collect motion sensor data in block 660. The patient's movement information is collected in block 670. The central controller 120 uses the motion sensor data and the selected pattern information to evaluate the patient's movement in block 670. In block 685, the patient's movement is compared with criteria pre-specified by the operator. If the patient completes a movement to the specified requirement, a positive feedback is created by the exercise assistance system in block 690, otherwise the process returns to block 680 to re-evaluate the patient's movement.

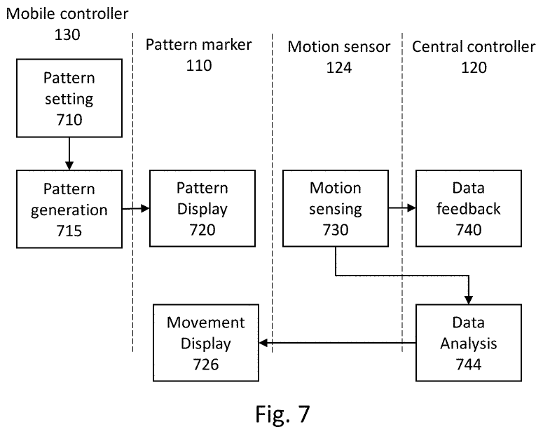

FIG. 7 is a block diagram illustrating a first exemplary operation of the assisted exercise system shown in FIG. 1. In this case, the mobile controller 130 performs pattern setting in block 710 and pattern generation in block 715. On the exercise ground, the pattern generated by the pattern maker 110 is displayed in block 720 and a patient's movement is also displayed in block 726. The motion sensor 124 senses the patient's location and movement in block 730. The patient's location and movement information are then provided to the central controller 120 as feedbacks in data feedback block 740 and for analysis in data analysis block 744.

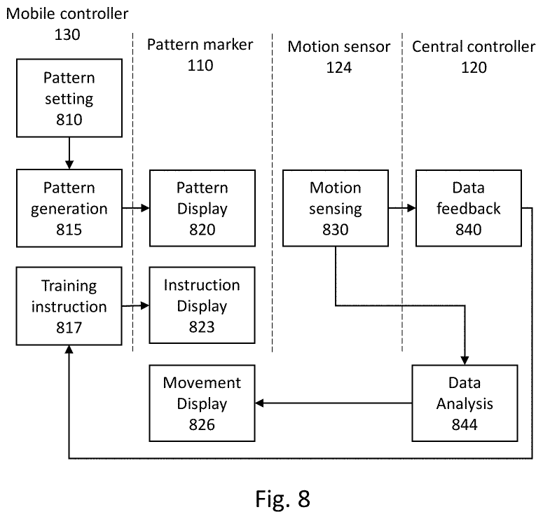

FIG. 8 is a block diagram illustrating a second exemplary operation of the assisted exercise system shown in FIG. 1. In this case, the mobile controller 130 performs pattern setting in block 810 and pattern generation in block 815. The mobile controller 130 also produces training instruction in block 817. The pattern generated by the pattern maker 110 is displayed on the exercise ground in block 820. The produced training instruction is also displayed on the exercise ground in block 823. At the same time, the patient's movement is also displayed on the exercise ground in block 826. The motion sensor detects the patent's location and movement in block 830. The motion data collected by the motion sensor 124 is then processed as data feedback in block 840 of the central controller 120. The feedback data is used to generate the training instruction by the mobile controller 130. The motion data collected by the motion sensor 124 is also provided to data analytic module of the central controller 120 in block 844. The data analytic module produces the patient movement data to be displayed in block 826.

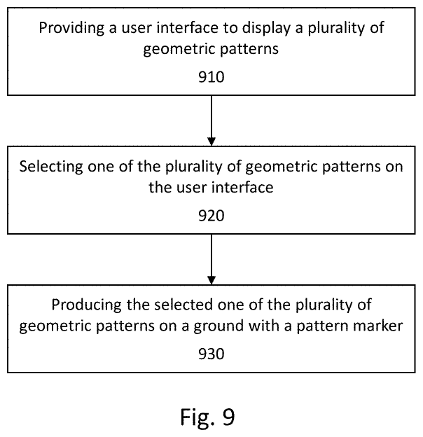

FIG. 9 is a flowchart illustrating a process of using the assisted exercise system shown in FIG. 1. The process begins with block 910 by providing a user interface to display a plurality of geometric patterns. In block 920, a user selects one of the plurality of geometric patterns on the user interface in block 920. In block 930, the assisted exercise system produces the selected one of the plurality of geometric patterns on a ground with a pattern marker for the user to begin an exercise session.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. The present disclosure can refer to the action and processes of a computer system, or similar electronic computing device, that manipulates and transforms data represented as physical (electronic) quantities within the computer system's registers and memories into other data similarly represented as physical quantities within the computer system memories or registers or other such information storage systems.

The present disclosure also relates to an apparatus for performing the operations herein. This apparatus can be specially constructed for the intended purposes, or it can include a general-purpose computer selectively activated or reconfigured by a computer program stored in the computer. Such a computer program can be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

The algorithms and displays presented herein are not inherently related to any particular computer or other apparatus. Various general-purpose systems can be used with programs in accordance with the teachings herein, or it can prove convenient to construct a more specialized apparatus to perform the method. The structure for a variety of these systems will appear as set forth in the description below. In addition, the present disclosure is not described with reference to any particular programming language. It will be appreciated that a variety of programming languages can be used to implement the teachings of the disclosure as described herein.

The present disclosure can be provided as a computer program product, or software, that can include a machine-readable medium having stored thereon instructions, which can be used to program a computer system (or other electronic devices) to perform a process according to the present disclosure. A machine-readable medium includes any mechanism for storing information in a form readable by a machine (e.g., a computer). In some embodiments, a machine-readable (e.g., computer-readable) medium includes a machine (e.g., a computer) readable storage medium such as a read only memory (“ROM”), random access memory (“RAM”), magnetic disk storage media, optical storage media, flash memory components, etc.

In this description, various functions and operations are described as being performed by or caused by computer instructions to simplify description. However, those skilled in the art will recognize what is meant by such expressions is that the functions result from execution of the computer instructions by one or more controllers or processors, such as a microprocessor. Alternatively, or in combination, the functions and operations can be implemented using special purpose circuitry, with or without software instructions, such as using Application-Specific Integrated Circuit (ASIC) or Field-Programmable Gate Array (FPGA). Embodiments can be implemented using hardwired circuitry without software instructions, or in combination with software instructions. Thus, the techniques are limited neither to any specific combination of hardware circuitry and software, nor to any particular source for the instructions executed by the data processing system.

Although the disclosure is illustrated and described herein as embodied in one or more specific examples, it is nevertheless not intended to be limited to the details shown, since various modifications and structural changes may be made therein without departing from the spirit of the disclosure and within the scope and range of equivalents of the claims. Accordingly, it is appropriate that the appended claims be construed broadly and, in a manner, consistent with the scope of the disclosure, as set forth in the following claims.

1. A physical exercise assistant system comprising:
a pattern generator configured to generate a plurality of geometric patterns;
a pattern marker coupled to the pattern generator and configured to produce a first one of the plurality of geometric patterns on a ground;
a user interface configured to display the plurality of geometric patterns and receive an input for selecting the first one of the plurality of geometric patterns; and
a motion sensor configured to detect an above-ground movement of an object within the first one of the plurality of geometric patterns, and provide detected movement data to the pattern generator and the user interface, wherein the object movement is selected from at least one of knee height, hips angle and center of gravity of the object.

2. The physical exercise assistant system of claim 1, wherein the plurality of geometric patterns is stored in a storage unit.

3. The physical exercise assistant system of claim 1, wherein the pattern generator is configured to alter a predetermined parameter of the first one of the plurality of geometric patterns.

4. The physical exercise assistant system of claim 3, wherein the predetermined parameter is selected from a set consisting of at least one of pattern category, pattern size, pattern position, pattern number and pattern color.

5. The physical exercise assistant system of claim 3, wherein the user interface is a mobile device with a touch panel display.

6. The physical exercise assistant system of claim 5, wherein the user interface is configured to create new geometric patterns for the pattern generator.

7. The physical exercise assistant system of claim 5, wherein the user interface is configured to invoke an instant change in the predetermined parameter of the first one of the plurality of geometric patterns in response to a user input.

8. The physical exercise assistant system of claim 1, wherein the pattern marker is a video projector.

9. The physical exercise assistant system of claim 1 further comprising a database storing an identification code and parameters associated with the first one of the plurality of geometric patterns, wherein the first one of the plurality of geometric patterns and the associated parameters can be selected by selecting the identification code.

10. The physical exercise assistant system of claim 9, wherein the motion sensor is configured for detecting an object location within a produced geometric pattern on the ground and providing the object location data to the pattern generator and the user interface.

11. The physical exercise assistant system of claim 10, wherein the object location is marked in the first one of the plurality of geometric patterns.

12. The physical exercise assistant system of claim 10 further comprising an analytic module configured to modify the parameters associated with the first one of the plurality of geometric patterns at least partially based on the object movement data.

13. The physical exercise assistant system of claim 12, wherein the analytic module is configured to switch the identification code to be associated with a second one of the plurality of geometric patterns at least partially based on the object movement data.

14. A physical exercise assistant method comprising:
providing a user interface to display a plurality of geometric patterns;
selecting a first one of the plurality of geometric patterns on the user interface;
producing the first one of the plurality of geometric patterns on a ground with a pattern marker;
detecting, by a motion sensor, an above-ground movement of an object within the first one of the plurality of geometric patterns, wherein the object movement is selected from at least one of knee height, hips angle and center of gravity of the object; and
providing detected movement data to the user interface.

15. The physical exercise assistant method of claim 14 further comprising altering a predetermined parameter of the first one of the plurality of geometric patterns.

16. The physical exercise assistant method of claim 15, wherein the predetermined parameter is selected from a set consisting of at least one of pattern category, pattern size, pattern position, pattern number and pattern color.

17. The physical exercise assistant method of claim 14, wherein the user interface is a mobile device with a touch panel display.

18. The physical exercise assistant method of claim 14 further comprising detecting an object location within the produced first one of the plurality of geometric patterns.

19. The physical exercise assistant method of claim 18 further comprising, at least partially based on the object movement data, modifying a parameter of the first one of the plurality of geometric patterns or selecting a second one of the plurality of geometric patterns.

20. A physical exercise assistant system comprising:
a central controller configured to generate a plurality of geometric patterns;
a pattern marker coupled to the central controller and configured to produce one of the plurality of geometric patterns on a ground;
a mobile controller configured to display the plurality of geometric patterns and receive an input for selecting the one of the plurality of geometric patterns; and
a motion sensor coupled to the central controller and configured to detect an object location and above-ground movement within the produced one of the plurality of geometric patterns and provide the object location and movement data to the central controller and the mobile controller, wherein the object movement is selected from at least one of knee height, hips angle and center of gravity of the object.
